(19) United States
(12) Patent Application Publication
(10) Pub. No.: US 2011/0152813 A1
(43) Pub. Date: Jun. 23, 2011

(54) Absorbent Article with Channel Portion

(76) Inventor: Daniel Lee Ellingson, Appleton, WI (US)

(21) Appl. No.: 12/640,626

(22) Filed: Dec. 17, 2009

(51) Int. Cl. A61F 13/15; A61F 13/45

(52) U.S. Cl. 604/374; 604/378; 604/385.23

(57) ABSTRACT

An absorbent article includes a liquid pervious body side liner, a liquid impervious outer cover, and an absorbent core located between the body side liner and the outer cover. The absorbent core includes a first absorbent portion, a second absorbent portion, and a channel portion. The channel portion extends the entire length of the absorbent core. The first absorbent portion, the second absorbent portion, and the channel portion all lie in the same plane. The channel portion completely separates the first absorbent portion from the second absorbent portion and the channel portion is a stabilized, high-loft material with permanent thermal bond points.

### BACKGROUND OF THE INVENTION

Traditional absorbent articles have typically included absorbent cores located between body side liners and outer covers. These articles have generally been used to absorb body exudates and the absorbent cores have frequently been comprised of cellulose fibers intermixed with superabsorbent polymers. These cores are often compressed to provide structural integrity. While these constructions have resulted in good performance and comfort, there still exists a need for absorbent articles having improved flexibility and conformance in use while still maintaining acceptable absorbent performance and fit.

### SUMMARY OF THE INVENTION

In response to these needs, the present invention provides an absorbent article having a liquid pervious body side liner, a liquid impervious outer cover, and an absorbent core located between the body side liner and the outer cover. The absorbent core has a first absorbent portion, a second absorbent portion, and a channel portion. The absorbent core defines a length in a longitudinal direction, a width in a lateral direction, and a thickness in a z-direction. The longitudinal direction and the lateral direction define a first plane. The channel portion extends the entire length of the absorbent core. The first absorbent portion, the second absorbent portion, and the channel portion all lie in the first plane. The channel portion completely separates the first absorbent portion from the second absorbent portion. The channel portion is a stabilized, high-loft material with permanent thermal bond points.

In various embodiments, the channel portion is a bonded carded web, a meltblown web, a coformed web, or a spunbonded web. In some embodiments, the channel portion is a bonded carded web made of hollow polypropylene fibers. In some embodiments, the channel portion is a powder bonded carded web made of hollow polyester fibers.

In various embodiments, the first absorbent portion and the second absorbent portion are air-laid. In some embodiments, the first absorbent portion and the second absorbent portion are made of a matrix of cellulosic fibers mixed with superabsorbent particles. In some embodiments, the channel portion is substantially free of absorbent material.

In some embodiments, the absorbent article also includes a surge material positioned between the body side liner and the first absorbent portion, the second absorbent portion, and the channel portion.

In some embodiments, the absorbent article also includes a core wrap having a first portion positioned between the body side liner and the absorbent core and a second portion positioned between the absorbent core and the outer cover. In various embodiments, the core wrap may be a nonwoven web made from meltblown thermoplastic fibers. In some embodiments, the absorbent core defines longitudinal edges and the core wrap is a unitary web that includes both the first portion and the second portion and the core wrap completely surrounds the longitudinal edges of the absorbent core. In some embodiments, the first portion and the second portion of the core wrap are adhesively joined to the first absorbent portion, the second absorbent portion, and the channel portion.

In another aspect, the present invention provides an absorbent article having a liquid pervious body side liner, a liquid impervious outer cover, and an absorbent core located between the body side liner and the outer cover. The absorbent core includes a first absorbent portion, a second absorbent portion, and a channel portion. The absorbent article defines a longitudinal direction, a lateral direction, and a z-direction and the longitudinal direction and the lateral direction define a first plane. The first absorbent portion, the second absorbent portion, and the channel portion all lie in the first plane. The channel portion extends the entire longitudinal direction and completely separates the first absorbent portion from the second absorbent portion. The first absorbent portion and the second absorbent portion are both air laid webs made of a matrix of cellulosic fibers mixed with superabsorbent particles. The channel portion is a bonded carded web, a meltblown web, a coformed web, or a spunbonded web.

In various embodiments, the channel portion is a bonded carded web made with hollow polypropylene fibers. In various embodiments, the channel portion is substantially free of absorbent material.

In some embodiments, the absorbent article also includes a surge material positioned between the body side liner and the first absorbent portion, the second absorbent portion, and the channel portion.

In some embodiments, the absorbent article also includes a core wrap having a first portion positioned between the body side liner and the absorbent core and a second portion positioned between the absorbent core and the outer cover.

In some embodiments, the channel portion also extends the entire lateral direction and completely separates a first piece of the first absorbent portion from a second piece of the first absorbent portion and completely separates a first piece of the second absorbent portion from a second piece of the second absorbent portion.

In another aspect, the present invention provides an absorbent article having a liquid pervious body side liner, a liquid impervious outer cover, and an absorbent core located between the body side liner and the outer cover. The absorbent core includes an absorbent portion, a first channel portion, and a second channel portion. The absorbent article defines a longitudinal direction, a lateral direction, and a z-direction and the longitudinal direction and the lateral direction define a first plane. The absorbent portion, the first channel portion, and the second channel portion all lie in the first plane. The first channel portion, the second channel portion, and the absorbent portion extend the entire longitudinal direction. The absorbent portion completely separates the first channel portion from the second channel portion. The first channel portion and the second channel portion are bonded carded webs.

In some embodiments, the bonded carded webs include hollow polypropylene fibers and the absorbent portion includes a matrix of cellulosic fibers mixed with superabsorbent particles.

### BRIEF DESCRIPTION OF THE DRAWINGS

FIG. 1 representatively illustrates a partially cut-away, top plan view of a representative absorbent article of the present invention in a stretched and laid flat condition with the surface that contacts the wearer facing the viewer.

FIG. 2 representatively illustrates a cross sectional view of the absorbent article of FIG. 1 taken along the line 2-2.

FIG. 3 representatively illustrates a perspective view of an exemplary absorbent core of the present invention.

FIG. 4 representatively illustrates a cross sectional view of an alternative absorbent article of the present invention.

FIG. 5 representatively illustrates a cross sectional view of an alternative absorbent article of the present invention.

FIG. 6 representatively illustrates a perspective view of another exemplary absorbent core of the present invention.

FIG. 7 representatively illustrates a cross sectional view of an alternative absorbent article of the present invention.

FIG. 8 representatively illustrates a perspective view of an exemplary absorbent core of the present invention.

### DETAILED DESCRIPTION OF THE DRAWINGS

The absorbent article of the present invention will be described in terms of a diaper adapted to be worn by infants about the lower torso. It is understood that the absorbent article of the present invention is equally applicable to other articles such as adult incontinent products, training pants, feminine care products, and the like.

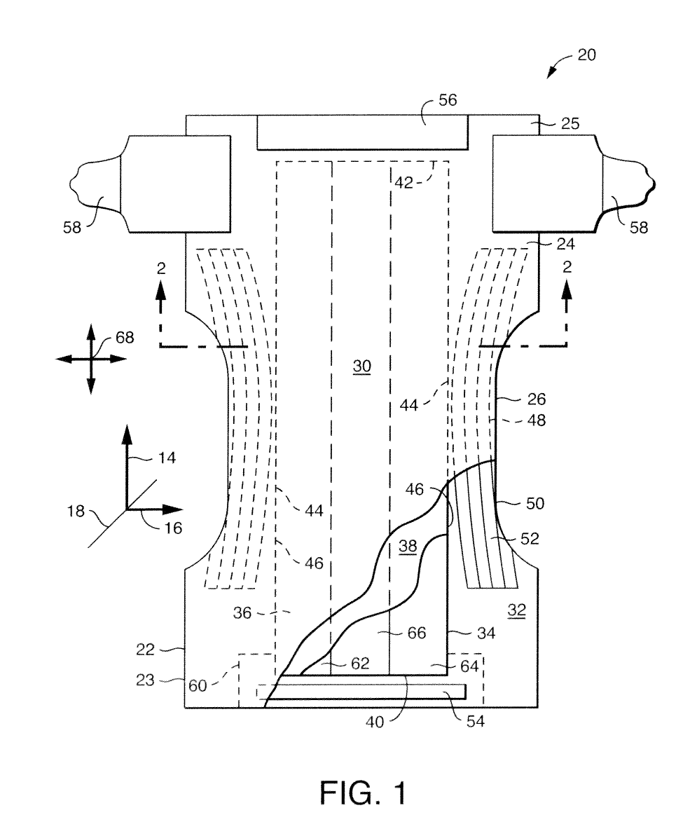

FIG. 1 representatively illustrates an embodiment of an absorbent article 20 of the present invention. The surface of the article which contacts the wearer is facing the viewer. The absorbent article 20 defines a front portion 22, a rear portion 24 and a crotch portion 26 connecting the front portion 22 and the rear portion 24. The front portion 22 defines a front waist region 23 and the rear portion 24 defines a rear waist region 25. The absorbent article 20 also defines a longitudinal direction 14 and a lateral direction 16. The absorbent article 20 includes a body side liner 30, an outer cover 32 and an absorbent assembly 34 located between the body side liner 30 and the outer cover 32.

The absorbent assembly 34 includes an absorbent core 36 and may include at least one core wrap 38. The absorbent core 36 has a front edge 40 and a rear edge 42. The front edge 40 and the rear edge 42 may be generally parallel and are opposed in the longitudinal direction 14. The absorbent core 36 also has laterally opposed side edges 44 and the core wrap 38 also has laterally opposed side edges 46.

As used herein, reference to a front portion refers to that part of the absorbent article which is generally located on the front of a wearer when in use. Reference to a front waist region refers to that part of the front portion which is located generally near the waist opening. Reference to the rear portion refers to the portion of the article generally located at the rear of the wearer when in use. Reference to a rear waist region refers to that part of the rear portion which is located generally near the waist opening. Reference to the crotch portion refers to that portion which is generally located between the legs of the wearer when in use.

The crotch portion 26 has opposite longitudinal side portions 48 which may include a pair of elasticized, longitudinally-extending leg cuffs 50. The leg cuffs 50 are generally adapted to fit about the legs of a wearer in use and serve as a mechanical barrier to the lateral flow of body exudates. The leg cuffs 50 may be elasticized by leg elastics 52. The absorbent article 20 may further include a front waist elastic 54 and/or a rear waist elastic 56.

The rear portion 24 of the absorbent article 20 may further include a fastening means 58 which is adapted to hold the absorbent article 20 about the waist of the wearer when in use. The fastening means 58 are typically joined to the rear portion 24 of the absorbent article 20 to provide a means for holding the article 20 on the wearer. Suitable fastening means 58 are well known to those skilled in the art and can include tape tab fasteners, hook and loop fasteners, mushroom and loop fasteners, snaps, pins, belts, and the like, and combinations thereof. Typically, the fastening means 58 are configured to be refastenable.

In some embodiments, the fastening means 58 may be adapted to engage or otherwise join with a fastener landing material 60. In some embodiments, the fastener landing material 60 is a loop material joined to the outer cover 32 in the front waist region 23 and adapted to engage hook-type fastening means 58. In other embodiments, the outer cover 32 may function as the fastener landing material 60 and may be adapted to engage hook-type fastening means 58. In yet other embodiments, the fastener landing material 60 may be a film adapted to engage with tape tab fastening means 58.

The absorbent article 20 may also include a pair of containment flaps which extend longitudinally along the absorbent article 20 and are also adapted to provide a barrier to the flow of body exudates. It should be recognized that individual components of the absorbent article 20, such as the elastic members, may be optional depending upon the intended use of the absorbent article 20.

The body side liner 30 of the absorbent article 20 suitably presents a body facing surface which is intended to be worn adjacent the body of the wearer and is compliant, soft feeling and nonirritating to the wearer's skin. Further, the body side liner 30 may be less hydrophilic than the absorbent assembly 34, to present a relatively dry surface to the wearer, and may be sufficiently porous to be liquid permeable, permitting liquid to readily penetrate through its thickness. A suitable body side liner 30 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The body side liner 30 is suitably employed to help isolate the wearer's skin from fluids held in the absorbent assembly 34.

Various woven and nonwoven fabrics can be used for the body side liner 30. For example, the body side liner may be composed of a meltblown or spunbonded web of polyolefin fibers. The body side liner may also be a bonded carded web composed of natural and/or synthetic fibers. The body side liner may be composed of a substantially hydrophobic material, and the hydrophobic material may, optionally, be treated with a surfactant or otherwise processed to impart a desired level of wettability and hydrophilicity.

The outer cover 32 of the absorbent article 20 may suitably be composed of a material which is either liquid permeable or liquid impermeable. It is generally preferred that the outer cover 32 be formed from a material which is substantially impermeable to fluids. For example, a typical outer cover can be manufactured from a thin plastic film or other flexible liquid-impermeable material. For example, the outer cover 32 may be formed from a polyethylene film. If it is desired to present the outer cover 32 with a more clothlike feeling, the outer cover 32 may comprise a polyethylene film having a nonwoven web laminated to the outer surface thereof, such as a spunbond web of polyolefin fibers. Methods of forming such clothlike outer covers are known to those skilled in the art.

Further, the outer cover 32 may be formed of a woven or nonwoven fibrous web layer which has been totally or partially constructed or treated to impart a desired level of liquid impermeability to selected regions that are adjacent or proximate the absorbent assembly 34. Still further, the outer cover 32 may optionally be composed of a micro-porous “breathable” material which permits vapors to escape from the composite absorbent assembly 34 while still preventing liquid exudates from passing through the outer cover 32.

The body side liner 30 and outer cover 32 are generally joined to one another so as to form a pocket in which the absorbent assembly 34 is located. The body side liner 30 and outer cover 32 may be joined directly to each other around the outer periphery of the absorbent article 20 by any means known to those skilled in the art, such as, for example, adhesive bonds, sonic bonds, thermal bonds, pressure bonds, and the like, and combinations thereof. For example, a uniform continuous layer of adhesive, a patterned layer of adhesive, a sprayed or meltblown pattern of adhesive or an array of lines, swirls or spots of adhesive may be used to join the body side liner 30 to the outer cover 32. In some embodiments, the outer cover 32 may include a full web spray of adhesive covering essentially the entire outer cover 32. The full web spray may be adapted to join the outer cover 32 with the body side liner 30 and/or the core wrap 38 and/or the absorbent core 36. Such bonding means may also be suitable for joining other components of the absorbent assembly and absorbent article of the present invention together.

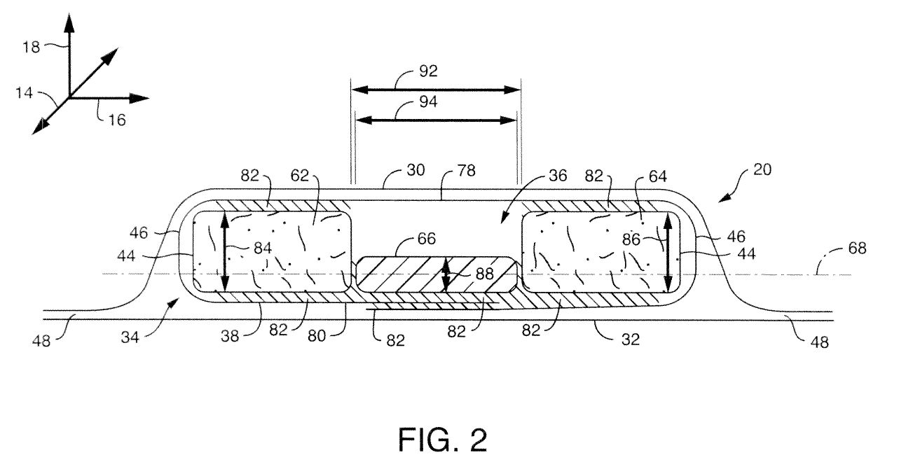

The absorbent assembly 34 is positioned between the body side liner 30 and the outer cover 32 to form the absorbent article 20. The absorbent assembly 34 is generally conformable and capable of absorbing and retaining body exudates. The absorbent assembly 34 and/or the absorbent core 36 may have any of a number of shapes and sizes. Referring now to FIG. 2, a cross sectional view of the absorbent article 20 of FIG. 1, taken along line 2-2, is representatively illustrated with the leg elastics removed for clarity.

As can be seen in FIG. 2, the absorbent article 20 includes the absorbent assembly 34 located between the body side liner 30 and the outer cover 32. The absorbent assembly 34 includes the absorbent core 36 and a first core wrap 38. The absorbent core 36 has laterally opposed side edges 44 and the core wrap 38 has laterally opposed side edges 46. The absorbent core 36 includes a first absorbent portion 62, a second absorbent portion 64, and a channel portion 66. The body side liner 30 and the outer cover 32 extend beyond the opposed side edges 44 and 46 to define the longitudinal side portions 48.

The absorbent article 20 also defines a z-direction 18 which is perpendicular to both the longitudinal direction 14 and the lateral direction 16. In some embodiments, the longitudinal direction 14 and the lateral direction 16 define a first plane 68. In the illustrated embodiment of FIG. 2, the first absorbent portion 62, the second absorbent portion 64, and the channel portion 66 all lie in the first plane 68.

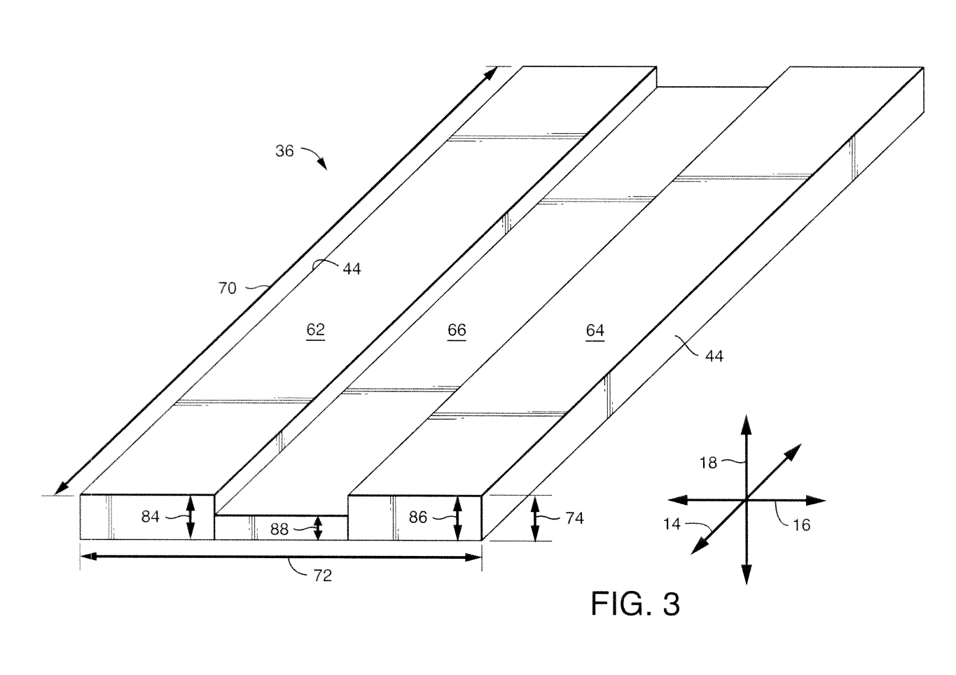

In various embodiments, the channel portion may extend the entire length of the absorbent core. For example, referring now to FIG. 3, a perspective view of an exemplary absorbent core 36 is illustrated. The absorbent core 36 defines a length 70 in the longitudinal direction 14, a width 72 in the lateral direction 16, and a thickness 74 in the z-direction 18 as illustrated. The channel portion 66 extends the entire length 70 of the absorbent core 36 and thus completely separates the first absorbent portion 62 from the second absorbent portion 64.

In some embodiments, the absorbent articles of the present invention may further include a surge material overlying the first absorbent portion, the second absorbent portion, and the channel portion. A surge portion is believed to advantageously improve the overall fluid intake rate of the absorbent core. The surge portion is typically less hydrophilic than the first absorbent portion 62 and the second absorbent portion 64 and is configured to collect and temporarily hold fluid surges. This configuration can also help prevent fluid exudates from pooling and collecting on portions of the absorbent core.

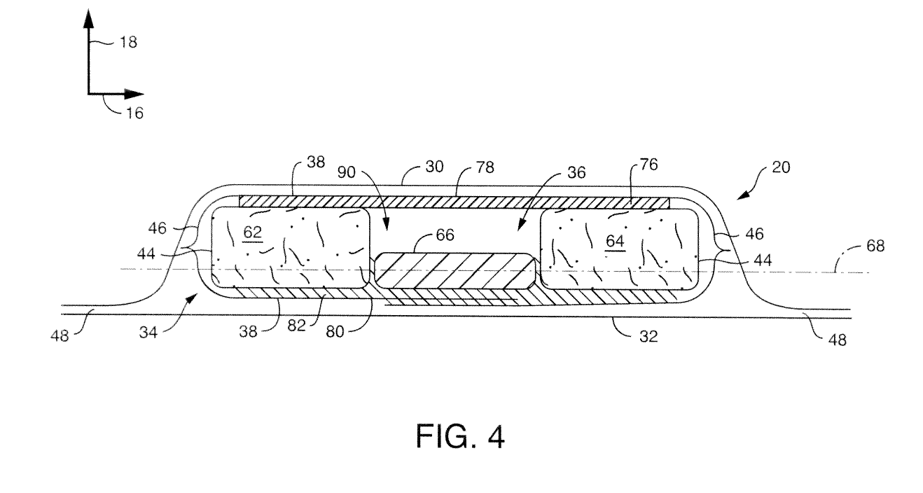

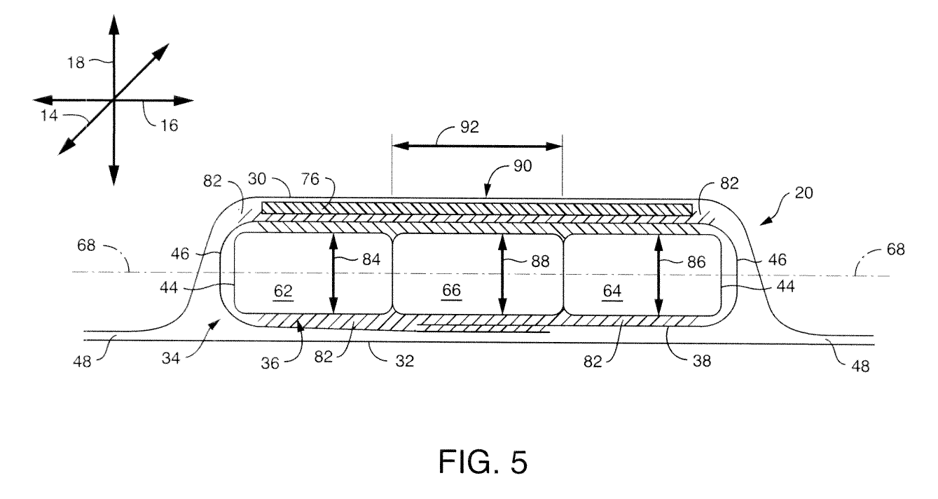

For example, FIG. 4 representatively illustrates an alternative cross sectional view of an exemplary absorbent article 20 that includes a surge material 76. In this embodiment, the surge material 76 is located between the core wrap 38 and the absorbent core 36. However, in some embodiments, the surge material 76 may be located between the core wrap 38 and the body side liner 30 as representatively illustrated in FIG. 5. In some embodiments, the absorbent assembly may not include a core wrap 38 such that the surge material 76 is located between the body side liner 30 and the absorbent core 36.

In some embodiments, the core wrap 38 may extend around the absorbent core 36 and define a first portion 78 generally positioned between the body side liner 30 and the absorbent core 36 and a second portion 80 generally positioned between the absorbent core 36 and the outer cover 32. In some embodiments, the core wrap 38 may include a single unitary material wrapped around the absorbent core 36 as illustrated in FIG. 2. In some embodiments, the core wrap 38 may include two or more materials wrapped around the absorbent core 36 as illustrated in FIG. 4.

In any of the arrangements, the core wrap 38 may be joined to itself, the surge material 76, the first absorbent portion 62, the second absorbent portion 64, the channel portion 66, the outer cover 32, the body side liner 30, or any other component of the absorbent article 20, or combinations thereof by any suitable means. For example, the core wrap 38 may be joined with adhesive 82 as illustrated in FIGS. 2 and 4. In some embodiments, the core wrap 38 may be joined to the first absorbent portion 62, the second absorbent portion 64 and the channel portion 66 on the liner side and the outer cover side with adhesive 82 as illustrated in FIG. 5.

In the various embodiments of the present invention, the first absorbent portion 62 defines a first dry absorbent thickness 84 in the z-direction 18, the second absorbent portion 64 defines a second dry absorbent thickness 86 in the z-direction 18, and the channel portion 66 defines a third dry thickness 88 in the z-direction 18 as representatively illustrated in FIG. 3. In various embodiments, the first dry absorbent thickness 84, the second dry absorbent thickness 86 and the third dry thickness 88 may be the same or may have different thicknesses. In some embodiments, the first dry absorbent thickness 84 is substantially the same as the second dry absorbent thickness 86 as illustrated in FIG. 3. In some embodiments, the third dry thickness 88 is less than 75%, less than 50%, or less than 35% of the first dry absorbent thickness 84 or the second dry absorbent thickness 86. For example, FIG. 2 representatively illustrates a channel portion 66 having a dry thickness 88 that is about 70% of the first dry absorbent thickness 84 of the first absorbent portion 64 and about 70% of the second dry absorbent thickness 86 of the second absorbent portion 66. While not wishing to be bound by theory, it is believed that the channel portion 66 having a third dry thickness 88 that is less than the first dry absorbent thickness 84 of the first absorbent portion 62 or the second dry absorbent thickness 86 of the second absorbent portion 64 provides additional void volume to receive body exudates. Additionally, it is also believed that the reduced thickness of the channel portion 66 provides increased conformance and flexibility of the absorbent article 20 to the body of the wearer.

However, in some embodiments, the third dry thickness 88 of the channel portion 66 may be greater than the first dry absorbent thickness 84 of the first absorbent portion 62 and may be greater than the second dry absorbent thickness 86 of the second absorbent portion 64 as illustrated in FIG. 5. Again, not wishing to be bound by theory, it is believed that channel portion 66 may still provide the absorbent article 20 with increased flexibility and conformance even in embodiments wherein the thickness of the channel portion 66 is equal to or greater than the first dry absorbent thickness 84 and/or the second dry absorbent thickness 86 based on proper selection of materials used in channel portion 66.

In some embodiments, the first absorbent portion 62 and the second absorbent portion 64 together define a channel 90 having a channel width 92 as measured in the lateral direction 16. In these embodiments, the channel portion 66 may have a width 94 that is less than or equal to the channel width 92 as illustrated in FIG. 2. In various embodiments, the channel 90 may completely separate the first absorbent portion 62 from the second absorbent portion 64.

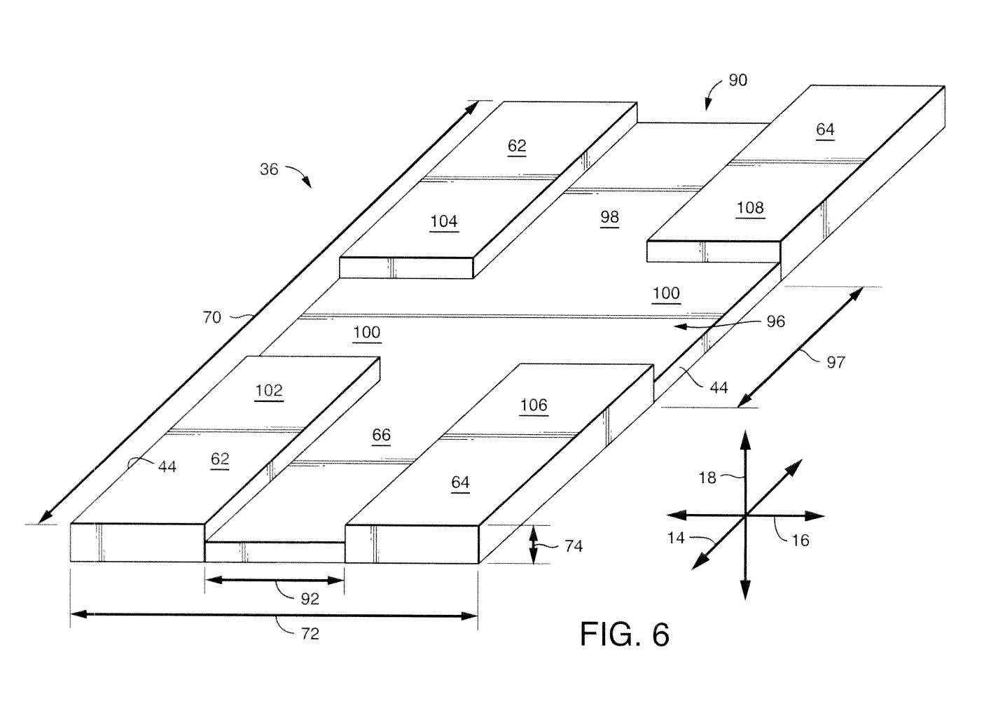

In some embodiments, the channel portion 66 may extend across part of the absorbent core. In some embodiments, the channel portion 66 may extend across the entire width of the absorbent core. For example, referring now to FIG. 6, a perspective view of an exemplary absorbent core 36 is illustrated. The absorbent core 36 defines a length 70 in the longitudinal direction 14, a width 72 in the lateral direction 16, and a thickness 74 in the z-direction 18. The channel portion 66 defines a longitudinal portion 98 and a lateral portion 100. The longitudinal portion 98 extends the entire length 70 of the absorbent core 36 and thus completely separates a first piece 102 of a first absorbent portion 62 from a first piece 106 of a second absorbent portion 64 and a second piece 104 of the first absorbent portion 62 from a second piece 108 of the second absorbent portion 64. Likewise, the lateral portion 100 extends the entire width 72 of the absorbent core 36 and thus completely separates the first piece 102 of the first absorbent portion 62 from the second piece 104 of the first absorbent portion 62 and the first piece 106 of the second absorbent portion 64 from the second piece 108 of the second absorbent portion 64. In this embodiment, the first piece 102 of the first absorbent portion 62, the second piece 104 of the first absorbent portion 62, the first piece 106 of the second absorbent portion 64, and the second piece 108 of the second absorbent portion 64 define a first channel 90 having a first channel width 92 as measured in the lateral direction 16 and a second channel 96 having a second channel width 97 as measured in the longitudinal direction 14. In these embodiments, the channel portion 66 may have a width that is less than or equal to the first channel width 92 and the second channel width 97 as illustrated in FIG. 6. In this embodiment, the first channel 90 and the second channel 96 completely separate the first piece 102 of the first absorbent portion 62, the first piece 106 of the second absorbent portion 64, the second piece 104 of the first absorbent portion 62, and the second piece 108 of the second absorbent portion 64.

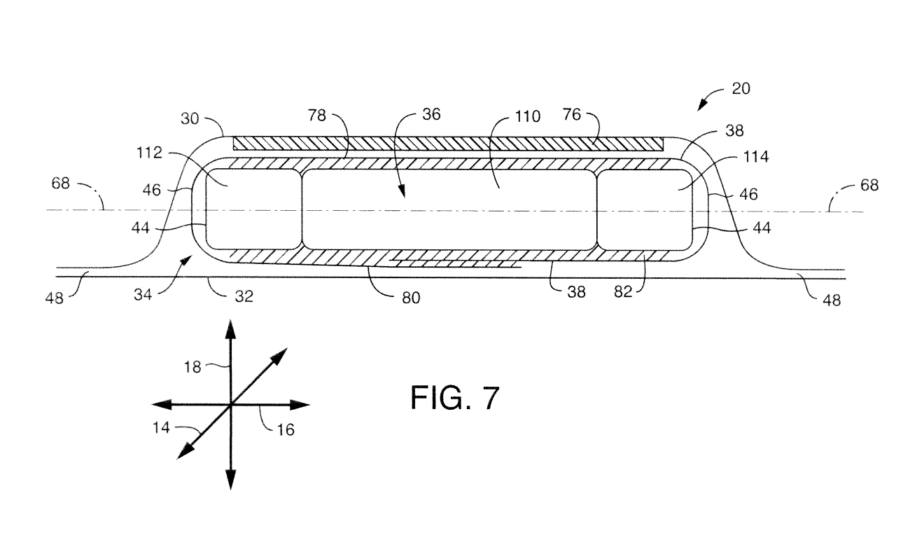

FIG. 7 representatively illustrates an alternative cross sectional view of an exemplary absorbent article 20. As can be seen in FIG. 7, the absorbent article 20 includes the absorbent assembly 34 located between the body side liner 30 and the outer cover 32. The absorbent assembly 34 includes the absorbent core 36 and a first core wrap 38. The absorbent core 36 has laterally opposed side edges 44 and the core wrap 38 has laterally opposed side edges 46. The absorbent core 36 includes an absorbent portion 110, a first channel portion 112, and a second channel portion 114. The body side liner 30 and the outer cover 32 extend beyond the opposed side edges 44 and 46 to define the longitudinal side portions 48. The absorbent article 20 also includes a surge material located between the body side liner 30 and the core wrap 38.

The absorbent article 20 also defines a z-direction 18 which is perpendicular to both the longitudinal direction 14 and the lateral direction 16. In some embodiments, the longitudinal direction 14 and the lateral direction 16 define a first plane 68. In the illustrated embodiment of FIG. 7, the absorbent portion 110, the first channel portion 112, and the second channel portion 114 all lie in the first plane 68.

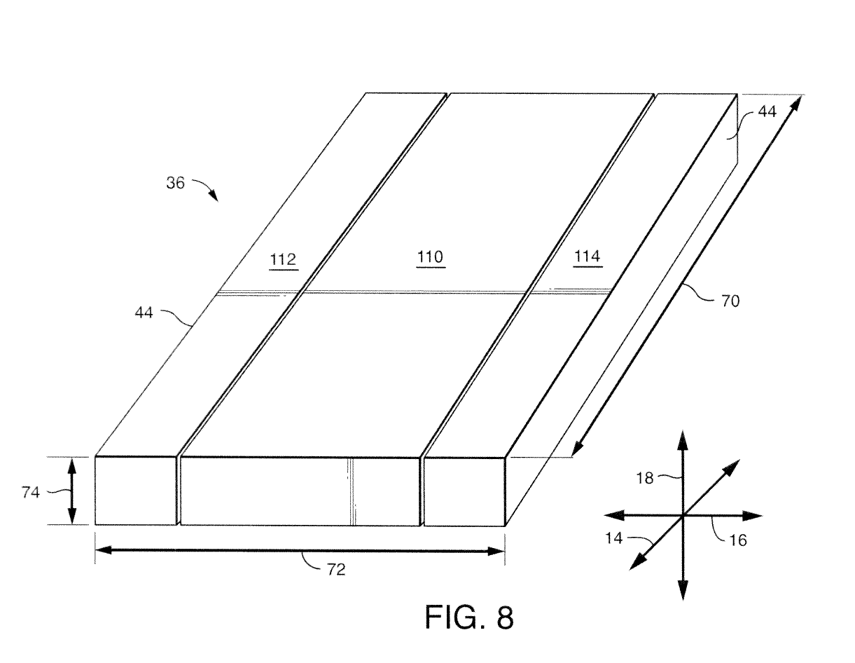

In various embodiments, the absorbent portion 110, the first channel portion 112, and the second channel portion 114 may extend the entire length of the absorbent core. For example, referring now to FIG. 8, a perspective view of an exemplary absorbent core 36 is illustrated. The absorbent core 36 defines a length 70 in the longitudinal direction 14, a width 72 in the lateral direction 16, and a thickness 74 in the z-direction 18. The absorbent portion 110, the first channel portion 112, and the second channel portion 114 extend the entire length 70 of the absorbent core 36. Thus, the absorbent portion 110 completely separates the first channel portion 112 from the second channel portion 114.

The absorbent portions 62, 64, 102, 104, 106, 108 and/or 110 may be made from materials or substances known in the art to absorb liquid as well as any others that may be developed for that purpose. For example, the absorbent portions may include fast and slow superabsorbent, pulps, foams, and mixtures thereof. In other examples, the absorbent portions may include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester and polyamide fibers; and synthetic fibers composed of a nonwettable thermoplastic polymer, such as polypropylene fibers, which have been hydrophilized by appropriate means known to those skilled in the art. The absorbent portions may also comprise selected blends of the various types of fibers mentioned above. The absorbent portions may include a matrix of hydrophilic fibers, such as a web of cellulosic fibers, mixed with particles of a high-absorbency material such as that commonly known as superabsorbent material. In some embodiments, the absorbent portions may include all cellulosic fibers. In other embodiments, the absorbent portion may include 30-90% by weight cellulosic fibers and 10-70% by weight superabsorbent material. In some embodiments, the absorbent portions may be air formed webs having 50-80% by weight cellulosic fibers and 20-50% superabsorbent material.

A “superabsorbent or superabsorbent material” refers to a water-swellable, water-soluble organic or inorganic material capable, under the most favorable conditions, of absorbing at least about 20 times its weight and, more desirably, at least about 30 times its weight in an aqueous solution containing 0.9 weight percent sodium chloride. Organic materials suitable for use as a superabsorbent material in conjunction with the present invention can include natural materials such as agar, pectin, guar gum, and the like; as well as synthetic materials, such as synthetic hydrogel polymers. Such hydrogel polymers include, for example, alkali metal salts of polyacrylic acids, polyacrylamides, polyvinyl alcohol, ethylene maleic anhydride copolymers, polyvinyl ethers, methyl cellulose, carboxymethyl cellulose, hydroxypropylcellulose, polyvinylmorpholinone; and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride polymers and mixtures thereof. The hydrogel polymers are preferably lightly crosslinked to render the materials substantially water insoluble. Crosslinking may, for example, be accomplished by irradiation or by covalent, ionic, van der Waals, or hydrogen bonding. The superabsorbent materials may be in any form suitable for use in absorbent composites including particles, fibers, flakes, spheres, and the like, and combinations thereof. Such superabsorbents are usually available in particle sizes ranging from about 20 to about 1000 microns. Any of the absorbent portions may have from 0 to 100 percent superabsorbent by weight based upon the total weight of the absorbent core. In various embodiments, any of the absorbent portions may have at least 30 percent, at least 40 percent, at least 50 percent, at least 60 percent, at least 70 percent, at least 80 percent or at least 90 percent superabsorbent material based on the total weight of the given absorbent portion.

Examples of suitable materials for the surge material 76 include various woven and nonwoven materials. For example, the surge material 76 may be a layer of a spunbonded or meltblown web of polyolefin fibers or a bonded carded web of natural and synthetic fibers. The surge material 76 may be a substantially hydrophobic material and, optionally, can be treated with a surfactant or otherwise to impart a desired level of wettability and hydrophilicity. The surge portion may be of any desired shape and configuration and the surge material 76 may have any suitable width in the lateral direction 16, any suitable length in the longitudinal direction 14, and any suitable thickness in the z-direction 18. Suitable surge materials 76 are taught in U.S. Pat. No. 5,429,629 to Latimer et al., the entirety of which is incorporated herein by reference to the extent consistent herewith.

In some embodiments, the channel portions 66, 112, and/or 114 may include various woven and nonwoven materials. For example, the channel portion may be a layer of a spunbonded or meltblown web of polyolefin fibers, a bonded carded web of natural and/or synthetic fibers, or coformed webs. The channel portion may be a substantially hydrophobic material and, optionally, can be treated with a surfactant or otherwise to impart a desired level of wettability and hydrophilicity. For example, the fibers may have contact angles less than 90°. In general, the channel portion is a stabilized, high-loft material having permanent thermal bond points.

Suitable synthetic polymers include, for example, polyethylene, polypropylene and polyesters. In some embodiments, the channel portion may be made from a plurality of fibers heat bonded to one another to form a lofty nonwoven web having any one or more of the following characteristics: a basis weight of at least 20 grams per square meter, a void volume of between about 80 and about 117 centimeters per gram of web while under a pressure of 689 dynes per square centimeter, a permeability of about 8,000 to about 15,000 darcy, a porosity of about 98.6 percent to about 99.4 percent; a surface area per void of about 10 to about 25 square centimeters per cubic centimeter; a saturation capacity of between about 55 and about 80 grams of 0.9 percent saline solution per gram of web; and/or a compression resilience in both the wet and dry state of at least about 60 percent. In some embodiments, the channel materials may be bonded carded webs having a basis weight of 50 gsm or more, 100 gsm or more, or 200 gsm or more and having densities of about 0.03 to about 0.04 g/cc. In some embodiments, the channel materials may be meltblown webs having a basis weight of 50 gsm or more, 100 gsm or more, or 200 gsm or more and having densities of about 0.07 to about 0.11 g/cc. In some embodiments, the channel materials may be pulp coform webs having a basis weight of 50 gsm or more, 100 gsm or more, or 200 gsm or more and having densities of about 0.03 to about 0.05 g/cc. In some embodiments, the channel materials may be staple coform webs having a basis weight of 180 gsm or more and having densities of about 0.02 to about 0.04 g/cc. Suitable channel materials having these characteristics are taught in U.S. Pat. No. 5,364,382 to Latimer et al., U.S. Pat. No. 5,429,629 to Latimer et al., and U.S. Pat. No. 5,486,166 to Bishop et al., the entireties of which are incorporated herein by reference to the extent consistent herewith. In various embodiments, the channel materials may include multiple layers of the various channel materials described herein. In various embodiments, the channel materials may include multiple layers to achieve the desired total basis weight.

In general, bonded carded webs are made from staple fibers which are typically provided in bales. The bales are placed in a picker which separates the fibers. Next, the fibers are sent through a combing or carding unit which further breaks apart and aligns the staple fibers in the machine direction so as to form a machine direction-oriented fibrous nonwoven web. Once the web has been formed and aligned, it is then bonded by one or more of several bonding methods.

The carded webs may be bonded with adhesive. For example, one bonding method is powder bonding wherein a powdered adhesive is distributed through the web and then activated, usually by heating the web and the adhesive with hot air. Another method for bonding includes through-air bonding, wherein heated air is forced through the web to melt and bond together the fibers at their crossover points. In some embodiments, the hot air may be approximately 143° C. and may have about a 4 second bonding time. Typically the unbonded web is supported on a forming wire or drum. In some embodiments, a vacuum may be pulled through the web to further contain the fibrous web during the bonding process.

In general, bonding processes such as point bonding and pattern bonding using smooth and/or pattern bonding rolls are less desirable because such processes will create a resultant fibrous nonwoven web which is too dense and does not have the degree of voids necessary for the channel material of the present invention. Whatever process is chosen, the degree of bonding will be dependent upon the fibers/polymers chosen but, in any event, it is desirable that there be as little compression as possible during the heating stage.

In one embodiment, the channel material may be a bonded carded web that includes a blend of polypropylene fibers and bicomponent polyethylene sheath/polyester core fibers. Specifically, the channel material of this embodiment may have a basis weight of about 50 gram per square meter (gsm) and may have a homogeneous blend of 60 percent by weight 4.4 denier by 38 millimeter polypropylene fibers from Danaklon a/s of Denmark and 40 weight percent 3.0 denier by 38 millimeter polyethylene sheath/polyester core bicomponent fibers from BASF Corporation Fibers Division of Enka, N.C. Both of these fibers may include a finish which makes the fibers hydrophilic. The two fibers may be uniformly mixed together, carded and then bonded using hot air at a temperature of approximately 143° C. for 4 seconds to bond the overall structure.

In another embodiment, the channel material may be a bonded carded web having a homogeneous blend of polyester fibers and polyethylene sheath/polyester core bicomponent fibers. Specifically, the channel material of this embodiment may have a basis weight of about 48 gsm and 40 percent by weight of a Hoechst Celanese type 295, 6.0 denier polyester fibers and 60 percent by weight of the BASF 3.0 denier polyethylene sheath/polyester core bicomponent fibers. The homogeneous blend of fibers may be bonded together using hot air at a temperature of 135° C. for approximately 4 seconds. During the bonding process, the samples may be compressed from an initial thickness of approximately 180-200 mils to a final heat-set thickness of approximately 100 mils (0.259 cm) at a pressure of 689 dynes per square centimeter.

In another embodiment, the channel material may be a powder-bonded-carded web. For example, the channel material may be composed of KODEL 435, 5.5 denier, polyester fibers bonded with EASTMAN 252 adhesive, which comprises about 16.6 wt % of the web. Channel materials of this embodiment may have a bulk density of about 0.1 g/cc, a bulk thickness of about 0.014 in, and a basis weight of about 30 g/yd².

In another embodiment, the channel material may be a spunbond web composed of polypropylene, trilobal fibers. In this embodiment, the channel material may have a bulk density of about 0.1 g/cc, a bulk thickness of about 0.017 in, and a basis weight of about 35 g/yd². The channel material of this embodiment may also include about 0-0.5 wt % of a selected surfactant, such as Triton X-102 distributed by Rohm & Haas Company of Philadelphia, Pa.

Other channel materials may include spunbond webs composed of polypropylene fibers, which may be round, trilobal or poly-lobal in cross-sectional shape and which may be hollow or solid in structure. Such webs can have a basis weight within the range of about 0.5-2.0 oz/yd² and a bulk thickness within the range of about 0.010-0.050 inch. Typically the webs are bonded, such as by thermal bonding, over about 3-30% of the web area.

In another embodiment, the channel material may include a powder-bonded-carded-web composed of hollow polyester fibers. For example, the channel material may be manufactured by H.D.K. located in Rogersville, Tenn. The H.D.K. material is composed of 100% polyester hollow fibers having a denier of about 5.5 and a bulk web density of about 0.02 g/cc. The web has a thickness of about 0.065 in (about 0.165 cm), a wet compression recovery value of 81% and a dry compression recovery value of about 88%. The web thickness is determined at a restraining pressure of 0.014 psi.

In another embodiment, the channel material may be powder-bonded-carded-web of polyester fibers. For example, the channel material may comprise a powder-bonded-carded-web composed of round polyester fibers having a denier of about 6. The fibers may be bonded with about 16 wt % of a polyester powder adhesive to form a web having a basis weight of about 30 g/yd², a bulk thickness of about 0.014 inch and a bulk density of approximately 0.10 g/cc and an average pore size of about 52 micrometers (ECD).

In another embodiment, the channel material may be a bonded carded web comprising 50 percent polyester fibers of 40 denier, 35 percent bleached cotton fibers of 1.5 denier and 15 percent Chisso ES fibers of 1.5 denier available from Chisso Corporation, Japan. The channel material of this embodiment may have a basis weight of about 211 gsm, a density of about 0.034 g/cc and may be treated with a solution of Triton® X102 surfactant (available from Rhom & Haas Co., Philadelphia, Pa.) by a dip and squeeze method to obtain a 0.5% surfactant add-on.

In another embodiment, the channel material may be a macro-fiber meltblown web having a 100 percent composition of a nylon-based polymer (e.g., Hydrofil® nylon by Allied Fibers Corporation of Morristown, N.J. By the term “macro-fiber meltblown”). The channel material of this embodiment, may have a mean fiber size of about 23 microns (micrometers) and a fiber diameter size distribution of about 3 to 100 microns, a basis weight of about 213 grams per meter² and a density of about 0.070 grams per centimeter³.

In some embodiments, the channel material may be a pulp coform material. For example, the channel material may be a blend of 50 percent cellulosic fluff (e.g., IP Supersoft from International Paper Corporation) and 50 percent macrofiber meltblown fibers of polypropylene (e.g., resin pellets from Himont U.S.A., Inc. of Wilmington, Del.). The channel material of this embodiment may be treated during formation with a solution of Triton X102 to obtain a 0.5% surfactant add-on. The channel material may have fiber sizes ranging from about 10-113 microns, an average fiber size of about 50 microns, a basis weight of 194 grams per meter², a density of 0.037 grams per centimeter³.

In some embodiments, the channel material may be a bonded carded web formed with about 40 wt % of 40 denier polyester fiber, about 25% of 3 denier rayon fiber, about 15% of 6.5 denier polyester fiber and about 20% of 6 denier Chisso ES fiber. The channel material of this embodiment may have a basis weight of 223 gsm and a density of 0.045 gm/cc.

In some embodiments, the channel material may be a staple coformed web of staple and melt extruded fibers. For example, the channel material may include 50 percent meltblown microfibers of Hydrofil® nylon. These fibers may have an average fiber diameter of 14 microns with a range of from 2 microns to 80 microns. The channel material of this embodiment may also include 38 percent polyester staple fibers of 25 denier and 12 percent Chisso ES fibers of 1.5 denier available from Chisso Corporation, Japan. The channel material of this embodiment may have a basis weight of 203 grams per meter² and a density of 0.038 grams per centimeter³.

In some embodiments, the channel material may be a staple coformed web of staple and melt extruded fibers. For example, the channel material may include 30 percent meltblown microfibers of polypropylene having a mean fiber diameter of 4 microns with a range of from 0.3 to 25 microns. The meltblown microfibers may then be blended with 56 percent polyester staple fibers of 25 denier (available, e.g., from E.I. Dupont de Nemours Corporation of Wilmington, Del.) and 14 percent Chisso ES fibers of 1.5 denier, available from Chisso Corporation, located in Japan. The channel material of this embodiment may have a basis weight of 183 grams per meter² and a density of 0.041 grams per centimeter³. Alternatively, the channel material of this embodiment may have a basis weight of 194 grams per meter² and a density of 0.023 grams per centimeter³.

In some embodiments, the channel material may be a 100 percent rayon spunbonded web such as the type sold by Futamura Chemicals, of Japan, under the tradename Taiko TCF. In these embodiments, the channel material may have a basis weight of 26 grams per meter² and a density of 0.112 grams per centimeter³.

In some embodiments, the channel material may be a bonded carded web made of bicomponent fibers and polyester fibers. For example, the channel material may be 60% by weight bicomponent fibers of 3 denier made with a polypropylene core and a polyethylene sheath. The bicomponent fibers are then combined with 40% by weight polyester fibers of 6 denier.

In some embodiments, the channel material may be a bonded carded web made of bicomponent fibers and hollow polypropylene fibers. For example, the channel material may include 60% by weight of bicomponent fibers of 3 denier made with a polypropylene core and polyethylene sheath. The bicomponent fibers may then be combined with 40% by weight hollow fiber polypropylene fibers of 7 denier.

Many of the channel materials disclosed herein include sheath/core bicomponent fibers. However, bicomponent fibers having other configurations (e.g., side by side) may be suitable in various embodiments. Additionally, any of the channel materials may be sealed or otherwise terminated at the longitudinal ends and/or lateral sides to restrict or stop fluid passage. For example, the channel materials may be terminated by crimping, twisting, or melting the channel fibers. In some embodiments, additional adhesive may be added to form a barrier to fluid passage.

The core wraps of the present invention may be a fibrous nonwoven web made from fine diameter thermoplastic fibers with particular pore sizes and air permeability. By thermoplastic fibers it is meant fibers which are formed from polymers such that the fibers can be bonded to themselves using heat or heat and pressure. While not being limited to the specific method of manufacture, meltblown fibrous nonwoven webs have been found to work particularly well. With respect to polymer selection, polyolefin fibers and especially polypropylene-based polymers have been found to work well. The fibers may be hydrophilic or hydrophobic, though it is desirable that one or more of the resultant core wraps be hydrophilic. As a result, the fibers may be treated to be hydrophilic as by the use of a surfactant treatment.

The core wraps may comprise fibers that are meltblown, spunbond, spunlace, spunbond-meltblown-spunbond, coform, or combinations thereof. The core wraps may have a significant amount of stretchability. For example, the structure of the core wraps may include an operative amount of elastomeric polymer fibers. Furthermore, the fibers utilized in the core wraps may be continuous or discontinuous.

The core wraps may comprise a stretchable, durable, hydrophilic, fluid pervious substrate. In some embodiments, the core wraps may comprise a coating including a hydrophilicity boosting amount of nanoparticles, wherein such nanoparticles have a particle size of from 1 to 750 nanometers. Examples of suitable nanoparticles include titanium dioxide, layered clay minerals, alumina oxide, silicates, and combinations thereof. Optionally, a nonionic surfactant can be added to the core wraps to provide additional or enhanced benefits.

In another aspect, the core wraps may be treated with a high-energy surface treatment. This high-energy treatment may occur prior to or concurrent with the hydrophilicity boosting composition coating described above. The high-energy treatment may be any suitable high-energy treatment for increasing the hydrophilicity of the core wrap. Suitable high-energy treatments include, but are not limited to, corona discharge treatment, plasma treatment, UV radiation, ion beam treatment, electron beam treatment and combinations thereof.

The core wraps may additionally or alternatively include materials such as surfactants, ion exchange resin particles, moisturizers, emollients, perfumes, natural fibers, synthetic fibers, fluid modifiers, odor control additives, lotions, viscosity modifiers, anti-adherence agent, pH control agents, and the like, and combinations thereof.

The core wraps may be in the form of films, nonwoven webs, and laminates of two or more substrates or webs. Additionally, the core wraps may be textured, apertured, creped, neck-stretched, heat activated, embossed, and micro-strained.

While the invention has been described in detail with respect to specific embodiments thereof, it will be appreciated that those skilled in the art, upon attaining understanding of the foregoing will readily appreciate alterations to, variations of, and equivalents to these embodiments. Accordingly, the scope of the present invention should be assessed as that of the appended claims and any equivalents thereto. Additionally, all combinations and/or sub-combinations of the disclosed embodiments, ranges, examples, and alternatives are also contemplated.

1. An absorbent article comprising,
a liquid pervious body side liner, a liquid impervious outer cover, and an absorbent core located between the body side liner and the outer cover,
the absorbent core comprising a first absorbent portion, a second absorbent portion, and a channel portion,
the absorbent core defining a length in a longitudinal direction, a width in a lateral direction, and a thickness in a z-direction, the longitudinal direction and the lateral direction defining a first plane, wherein
the first absorbent portion, the second absorbent portion, and the channel portion all lie in the first plane;
the channel portion extends the entire length of the absorbent core;
the channel portion completely separates the first absorbent portion from the second absorbent portion; and
the channel portion is a stabilized, high-loft material with permanent thermal bond points.

2. The absorbent article of claim 1 wherein the channel portion is a bonded carded web, a meltblown web, a coformed web, or a spunbonded web.

3. The absorbent article of claim 1 wherein the channel portion is a bonded carded web comprising hollow polypropylene fibers.

4. The absorbent article of claim 1 wherein the channel portion is a powder bonded carded web comprising hollow polyester fibers.

5. The absorbent article of claim 1 wherein the first absorbent portion and the second absorbent portion are air-laid.

6. The absorbent article of claim 5 wherein the first absorbent portion and the second absorbent portion comprise a matrix of cellulosic fibers mixed with superabsorbent particles.

7. The absorbent article of claim 6 wherein the channel portion is substantially free of absorbent material.

8. The absorbent article of claim 7 further comprising a surge material positioned between the body side liner and the first absorbent portion, the second absorbent portion, and the channel portion.

9. The absorbent article of claim 1 wherein the absorbent article further comprises a core wrap having a first portion positioned between the body side liner and the absorbent core and a second portion positioned between the absorbent core and the outer cover.

10. The absorbent article of claim 9 wherein the core wrap is nonwoven web made from meltblown thermoplastic fibers.

11. The absorbent article of claim 10 wherein the absorbent core defines longitudinal edges and the core wrap is a unitary web comprising both the first portion and the second portion and the core wrap completely surrounds the longitudinal edges of the absorbent core.

12. The absorbent article of claim 11 wherein the first portion and the second portion are adhesively joined to first absorbent portion, the second absorbent portion, and the channel portion.

13. An absorbent article comprising,
a liquid pervious body side liner, a liquid impervious outer cover, and an absorbent core located between the body side liner and the outer cover,
the absorbent core comprising a first absorbent portion, a second absorbent portion, and a channel portion,
the absorbent article defining a longitudinal direction, a lateral direction, and a z-direction, the longitudinal direction and the lateral direction defining a first plane, wherein
the first absorbent portion, the second absorbent portion, and the channel portion all lie in the first plane;
the channel portion extends the entire longitudinal direction and completely separates the first absorbent portion from the second absorbent portion;
the first absorbent portion and the second absorbent portion are both air laid webs comprising a matrix of cellulosic fibers mixed with superabsorbent particles; and
the channel portion is a bonded carded web, a meltblown web, a coformed web, or a spunbonded web.

14. The absorbent article of claim 13 wherein the channel portion is a bonded carded web comprising hollow polypropylene fibers or hollow polyester fibers.

15. The absorbent article of claim 13 wherein the channel portion is substantially free of absorbent material.

16. The absorbent article of claim 13 further comprising a surge material positioned between the body side liner and the first absorbent portion, the second absorbent portion, and the channel portion.

17. The absorbent article of claim 13 wherein the absorbent article further comprises a core wrap having a first portion positioned between the body side liner and the absorbent core and a second portion positioned between the absorbent core and the outer cover.

18. The absorbent article of claim 13 wherein the channel portion extends the entire lateral direction and completely separates a first piece of the first absorbent portion from a second piece of the first absorbent portion and completely separates a first piece of the second absorbent portion from a second piece of the second absorbent portion.

19. An absorbent article comprising,
a liquid pervious body side liner, a liquid impervious outer cover, and an absorbent core located between the body side liner and the outer cover,
the absorbent core comprising an absorbent portion, a first channel portion, and a second channel portion,
the absorbent article defining a longitudinal direction, a lateral direction, and a z-direction, the longitudinal direction and the lateral direction defining a first plane, wherein
the first channel portion, the second channel portion, and the absorbent portion extend the entire longitudinal direction;
the absorbent portion completely separates the first channel portion from the second channel portion;
the absorbent portion, the first channel portion, and the second channel portion all lie in the first plane; and
the first channel portion and the second channel portion are bonded carded webs.

20. The absorbent article of claim 19 wherein the bonded carded webs comprise hollow polypropylene fibers and the absorbent portion comprises a matrix of cellulosic fibers mixed with superabsorbent particles.
